(19) United States
(12) Patent Application Publication
(10) Pub. No.: US 2023/0333101 A1
(43) Pub. Date: Oct. 19, 2023

(54) CONTINUOUS OPTICAL APTAMER SENSORS

(71) Applicant: University Of Cincinnati, Cincinnati, OH (US)

(72) Inventor: Jason Charles Heikenfeld, Cincinnati, OH (US)

(21) Appl. No.: 18/027,393

(22) PCT Filed: Sep. 24, 2021
(86) PCT No.: PCT/US2021/051967

§ 371 Date: Mar. 21, 2023

Related U.S. Application Data
(60) Provisional application No. 63/197,674, filed on Jun. 7, 2021.
(60) Provisional application No. 63/150,667, filed on Feb. 18, 2021.
(60) Provisional application No. 63/150,677, filed on Feb. 18, 2021.
(60) Provisional application No. 63/150,712, filed on Feb. 18, 2021.
(60) Provisional application No. 63/150,856, filed on Feb. 18, 2021.
(60) Provisional application No. 63/150,865, filed on Feb. 18, 2021.
(60) Provisional application No. 63/150,894, filed on Feb. 18, 2021.
(60) Provisional application No. 63/150,944, filed on Feb. 18, 2021.
(60) Provisional application No. 63/150,153, filed on Feb. 17, 2021.
(60) Provisional application No. 63/150,986, filed on Feb. 18, 2021.
(60) Provisional application No. 63/136,262, filed on Jan. 12, 2021.
(60) Provisional application No. 63/122,071, filed on Dec. 7, 2020.
(60) Provisional application No. 63/122,076, filed on Dec. 7, 2020.
(60) Provisional application No. 63/085,484, filed on Sep. 30, 2020.
(60) Provisional application No. 63/082,834, filed on Sep. 24, 2020.
(60) Provisional application No. 63/082,999, filed on Sep. 24, 2020.
(60) Provisional application No. 63/083,029, filed on Sep. 24, 2020.

(51) Int. Cl. G01N 33/543; G01N 33/58; G01N 33/542; G01N 27/327

(52) U.S. Cl. CPC G01N 33/5438; G01N 33/582; G01N 33/542; G01N 27/3275

(57) ABSTRACT

A device for detecting at least one analyte in a sample fluid is provided. The device 100 includes a sensor fluid 18, a plurality of aptamers disposed in the sensor fluid, one or more aptamers of the plurality of aptamers configured to bind to an analyte, an optical source 120, and an optical detector 122 configured to detect a change in at least one optical property of the aptamers, and at least one isolation element (e.g., membrane 136) retaining the aptamer in the sensor fluid. Each of the one or more aptamers includes at least one optical tag. Each of the optical tags is configured to provide a change in at least one optical property between a first state in which the aptamer is bound to the analyte and a second state in which the aptamer is not bound to the analyte, and the first state and the second state differing in the shape of the aptamer.

### CROSS-REFERENCE TO RELATED APPLICATIONS

This application claims the benefit of the filing date of U.S. patent application Ser. No. 63/082,834, filed on Sep. 24, 2020; claims the benefit of the filing date of U.S. patent application Ser. No. 63/082,999, filed on Sep. 24, 2020; claims the benefit of the filing date of U.S. patent application Ser. No. 63/083,029, filed on Sep. 24, 2020; claims the benefit of the filing date of U.S. patent application Ser. No. 63/085,484, filed on Sep. 30, 2020; claims the benefit of the filing date of U.S. patent application Ser. No. 63/122,071, filed on Dec. 7, 2020; claims the benefit of the filing date of U.S. patent application Ser. No. 63/122,076, filed on Dec. 7, 2020; claims the benefit of the filing date of U.S. patent application Ser. No. 63/136,262, filed on Jan. 12, 2021; claims the benefit of the filing date of U.S. patent application Ser. No. 63/150,667, filed on Feb. 18, 2021; claims the benefit of the filing date of U.S. patent application Ser. No. 63/150,677, filed on Feb. 18, 2021; claims the benefit of the filing date of U.S. patent application Ser. No. 63/150,712, filed on Feb. 18, 2021; claims the benefit of the filing date of U.S. patent application Ser. No. 63/150,856, filed on Feb. 18, 2021; claims the benefit of the filing date of U.S. patent application Ser. No. 63/150,865, filed on Feb. 18, 2021; claims the benefit of the filing date of U.S. patent application Ser. No. 63/150,894, filed on Feb. 18, 2021; claims the benefit of the filing date of U.S. patent application Ser. No. 63/150,944, filed on Feb. 18, 2021; claims the benefit of the filing date of U.S. patent application Ser. No. 63/150,953, filed on Feb. 18, 2021; claims the benefit of the filing date of U.S. patent application Ser. No. 63/150,986, filed on Feb. 18, 2021; and claims the benefit of the filing date of U.S. patent application Ser. No. 63/197,674, filed on Jun. 7, 2021, the disclosures of each of which are incorporated by reference herein in their entireties.

### TECHNICAL FIELD

The present invention relates to the use of aptamer sensors.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Aptamers are nucleic acid or peptide molecules that bind to a target molecule with high specificity. After selection and enrichment, aptamers possess similar affinities to antibody-antigen pairs, but have the advantage of being able to be synthesized using standard methods. As synthetic molecules, aptamers also have unique advantages in the control of their size and their amenability for chemical modification and, as such, have been widely developed and applied in the development of sensors. Electrochemical, aptamer-based (EAB) sensors have emerged in recent years as a platform to detect proteins, small molecules, and inorganic ions, relying on the induced conformational change of oligonucleotide aptamers in the presence of specific analyte. When a target molecule binds to an aptamer, which may be tethered to an electrode surface, changes in the aptamer structure are measured by changes in the electrochemical signal of an attached redox label on the aptamer. EAB sensor technology presents a stable, reliable, bioelectric sensor that is sensitive to the target analyte in a sample, while being capable of multiple analyte capture events during the sensor lifespan.

At least one application of EAB sensors is continuous biosensing. While continuous biosensing has seen success primarily with glucose monitoring for diabetes, there has been little success beyond such applications. One of the fundamental challenges facing continuous biosensors is lifetime of the sensors, for which glucose-oxidase electrodes, an enzymatic electrode sensor, can currently provide up to 2 weeks of operation. In such glucose oxidase electrodes, these electrode surfaces need to be simply close to the electrically interrogating electrochemical electrode. However, that surface may change, for example by the presence of a fouling species, over time and the device will still operate. The challenge with glucose and other enzymatic sensors is that they are not generalizable. That is, unlike aptamers which are developed rapidly for a target analyte using SELEX technology, enzymes used in enzymatic sensors are not easily developed for any target analyte.

In contrast to enzymatic sensors, aptamer sensors are an emerging class of sensors that are highly generalizable, but they have their own drawbacks. The only truly continuous aptamer based sensors are those based on attachment of aptamers to an electrochemical electrode, which brings about lifetime challenges as the surface of that electrode is subject to degradation and fouling over time. Others have shown continuous use of ‘molecular beacon’ aptamers that are optical based, but these may not be suitable for continuous use for biosensing applications because they are conducted, for example, in a Petri dish. Such an example is not really a biosensor. Rather, such an arrangement is akin to a human having optical aptamers continuously injected into their blood and then fluorescently measured in-vivo. Rather, optical aptamers, to date, have not been included in any sensing device; instead, they are known only to have use as a stand-alone material, used in, for example, benchtop assay tests. This is unfortunate, because optical aptamer sensors could potentially have lifetimes that extend far beyond the current alternative sensor lifetimes because aptamer sensors need not be placed on an electrode surface. Therefore, a need still exists for devices that fully enable continuous sensing with optical aptamers.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with sample fluids containing at least one analyte of interest to be measured.

Various aspects of the disclosed invention are directed to continuous aptamer sensors with previously unachievable performance by placing the aptamer in the solution phase with an architecture for the aptamer, and with cleaning strategies for electrodes that would otherwise foul or fully-passivate over long usage periods.

One particular aspect is directed to a continuous sensing device for sensing at least one analyte from a sample fluid is provided. The device includes a sensor fluid and a plurality of aptamers disposed in the sensor fluid. One or more aptamers of the plurality of aptamers is configured to bind to an analyte. Each of the aptamers includes at least one optical tag, and the optical tag is configured to provide a change in at least one optical property between a first state in which the aptamer is bound to the analyte and a second state in which the aptamer is not bound to the analyte. The shape of the aptamer differs in first state and the second state. The device further includes at least one isolation element retaining the aptamer in the sensor fluid, an optical source configured to emit light, the optical source in communication with the sensor fluid, and an optical detector configured to detect a change in at least one optical property of the aptamers.

Another particular aspect is directed to a method of sensing an analyte in a sample solution is also provided. The method includes bringing a sample fluid into contact with a plurality of aptamers in a sensor fluid. One or more aptamers of the plurality of aptamers includes a fluorescent tag and a quencher. The fluorescent tag is configured to emit an amount of light, and the quencher is configured to quench at least a portion of the light emitted by the fluorescent tag, the aptamer is configured to shift between an open state in which the aptamer is bound to the analyte and a closed state in which the aptamer is not bound to the analyte. Furthermore, the aptamer configured to emit more light in the open state than in the closed state. The method further includes binding the analyte included in the sample solution to the aptamer resulting in an increase in fluorescence emitted by the aptamer. The method further includes detecting the increased fluorescence emitted by the aptamer.

### BRIEF DESCRIPTION OF THE DRAWINGS

The objects and advantages of the disclosed invention will be further appreciated in light of the following detailed descriptions and drawings in which:

FIG. 1 is a cross-sectional view of a device according to a conventional aptamer sensor device.

FIG. 2 is a cross-sectional view of a device according to an embodiment of the disclosed invention.

FIGS. 3A-C are schematic views of example aptamers of the disclosed invention.

FIG. 4 is plot of fluorescence lifetime and therefore intensity as a function of distance between an optical fluorescent tag and a quencher.

FIG. 5 is a schematic of an embodiment of a device in accordance with principles of the present invention.

### DEFINITIONS

As used herein, “continuous sensing” with a “continuous sensor” means a sensor that changes in response to changing concentration of at least one solute in a solution such as an analyte. Similarly, as used herein, “continuous monitoring” means the capability of a device to provide multiple measurements of an analyte over time.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “aptamer” means a molecule that undergoes a conformation or binding change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function but which behave analogous to traditional aptamers. Two or more aptamers bound together can also be referred to as an aptamer (i.e. not separated in solution). Aptamers can have molecular weights of at least 1 kDa, 10 kDa, or 100 kDa.

As used herein, the term “change in electron transfer” means a redox tag whose electron transfer with an electrode has changed in a measurable manner. This change in electron transfer can, for example, originate from availability for electron transfer, distance from an electrode, diffusion rate to or from an electrode, a shift or increase or decrease in electrochemical activity of the redox tag, or any other embodiment as taught herein that results in a measurable change in electron transfer between the redox tag and the electrode.

As used herein, the term “fluorescent tag”, “tag”, and “fluorescent quencher”, and quencher means molecules which are like those used in molecular beacon laboratory assays. Examples of fluorescent tags include 6-FAM (carboxylfluorescein), JOE, TET, HEX, and examples of quenchers include black-hole quenchers, DABCYL. These tags may also be referred to as “optical tags” more generally, as there are multiple types of optical emission beyond fluorescence such as phosphorescence, and because other optical properties such as optical absorbance magnitude or peak wavelength for optical absorption can also be measurable aspects of the tags.

As used herein, the term “folded aptamer” means an aptamer that along its length associates with itself in one or more locations creating a three-dimensional structure for the aptamer that is distinct from an “unfolded aptamer” that is a freely floating and oscillating strand of aptamer. Aptamers can also be partially folded or partially unfolded in structure or in time spent in the folded vs. unfolded states. Multiple folding configurations are also possible.

As used herein, the term “analyte” means any solute in a solution or fluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a solution or fluid.

As used herein, the term “membrane” means a polymer film, plug of hydrogel, liquid-infused film, tiny pore, or other suitable material which is permiselective to transport of a solute through the membrane by solute parameters such as size, charge state, hydrophobicity, physical structure, or other solute parameters than can enable permiselectivity. For example a dialysis membrane is permselective by passing small solutes but not large solutes such as proteins. Membranes as understood herein need not be multiporous, for example a nanotube or nanopore can act as a permiselective filter and is therefore considered part of a membrane as understood for the present invention. Permiselectivity can scale with the analyte, for example a membrane with a molecular weight cut-off of 50 kDa could be used to measure a 20-30 kDa protein but could still keep out cellular or other large content (globulins, fibrogen, etc.) and retain in aptamer that adequately large or physically structured such that permeability through the membrane is slow or nil.

As used herein, the term “sample fluid” means any solution or fluid that contains at least one analyte to be measured.

As used herein, the term “sensor fluid” means a solution or fluid that differs from a sample solution by at least one property, and through which the sensor solution and the sample solution are therefore separated but are in fluidic connection through at least one pathway such as a membrane. The sensor solution comprises at least one aptamer as a solute.

As used herein, the term “reservoir fluid” means a solution or fluid that differs from a sample solution by at least one property, and through which the sensor solution and the reservoir solution are in fluidic connection through at least one pathway such as a membrane or a pin-hole connection. A reservoir fluid may have multiple function in a device, for example, by introducing a solute continuously or as needed by diffusion equilibrium into the sensor fluid, or for example removing unwanted solutes from a sensor fluid and acting as a “waste removal element”.

As used herein, a “device” comprises at least one sensor based on at least one aptamer, at least one sensor solution, and at least one sample solution. Devices can sense multiple samples and be in multiple configurations such as a device to measure a pin-prick of blood, or a microneedle or in-dwelling sensor needle to measure interstitial fluid, or a device to measure saliva, tears, sweat, or urine sensor, or a device to measure water pollutants or food processing solutes, or other devices which measure at least one analyte found in a sample solution.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors measure a characteristic of an analyte. Sensors are preferably electrical in nature, but may also include optical, chemical, mechanical, or other known biosensing mechanisms. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a battery, antenna, adhesive), and for purposes of brevity and focus on inventive aspects, such components may not be explicitly shown in the diagrams or described in the embodiments of the disclosed invention. All ranges of parameters disclosed herein include the endpoints of the ranges.

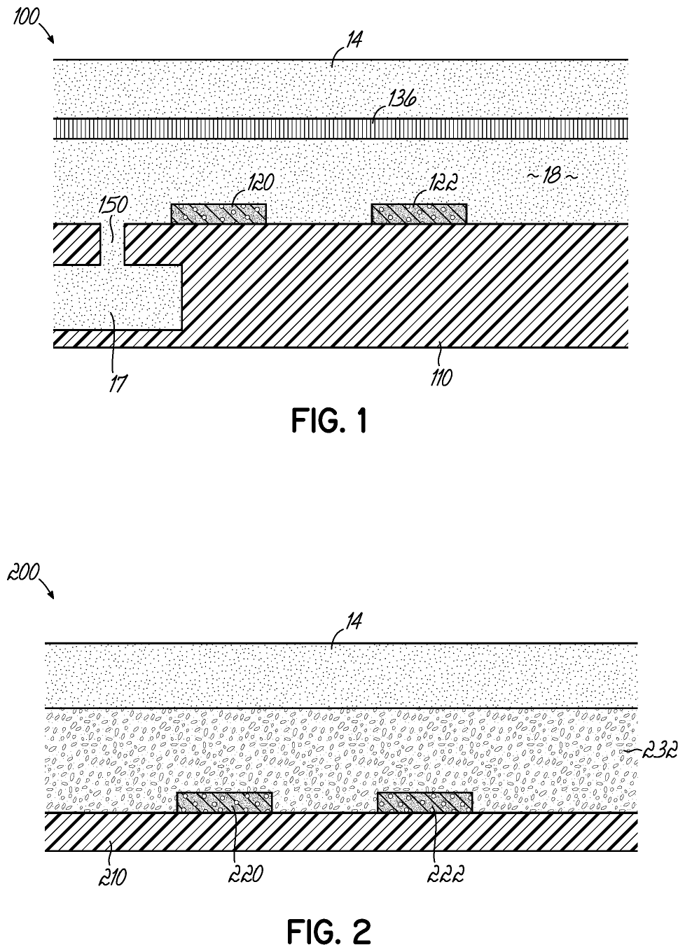

With reference to FIG. 1, a device 100 includes a sample fluid 14 such as, in one embodiment, interstitial fluid, a membrane 136 such as, in one embodiment, a 200-500 Da molecular weight cutoff dialysis membrane, a sensor fluid 18, an optical source 120 such as, in one embodiment, an LED to excite fluorescence and an optical detector 122 such as, in one embodiment, a photodiode with an optical filter to detect fluorescence and a substrate 110. The membrane 136 acts as an isolation element in device 100 in that species included in sample fluid 14 must first permeate the membrane 136 prior to entering the sensor fluid 18 or potentially being exposed to the optical source 120 or the optical detector 122. Sensor fluid 18 is also connected by a diffusion limited pathway 150 to a reservoir fluid 17, which allows sensor fluid 18 or waste generated, from for example use of the device 100, to be removed from the sensor fluid 18 and segregated to the reservoir fluid 17. As shown in FIG. 1, the sensor fluid 18 is in fluid communication with the reservoir fluid 17.

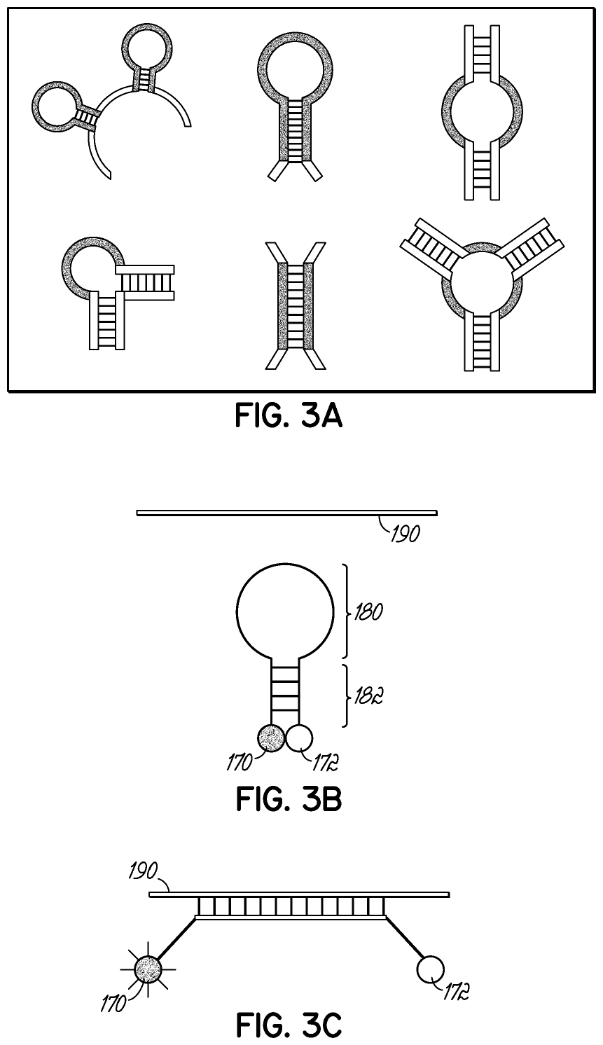

The sensor fluid 18 contains a plurality of aptamers that are configured to specifically bind to at least one analyte 190 (shown in FIGS. 3B and 3C) from the sample fluid 14. The optical source 120 is configured to emit wavelengths of light to stimulate a fluorescent tag 170 (shown in FIGS. 3B and 3C) located on each aptamer of the plurality of aptamers. The aptamers are retained in a location proximal to the optical source 120 and optical detector 122 by the membrane 136 and the substrate 110 (in the embodiment illustrated in FIG. 1), using, in one embodiment, size-selective permeability of the membrane 136 that retains the aptamers (typically, the aptamers are sized between 1 kDa and 10s of kDa) while allowing analytes (such as analyte 190) initially present in the sample fluid 14 (sized, for example, at 100s of Da) to pass through the membrane 136 (as a non-limiting example, the analyte 190 may be a drug such as tenofovir). The aptamer is optically tagged with a fluorescent tag 170 and optically tagged with a quencher 172 (shown in FIGS. 3B and 3C) that change in proximity to each other, and therefore fluorescent tag 170 is able to fluoresce when moved away from quencher 172, as is employed in molecular beacon aptamer technologies. Both the fluorescent tag 170 and the quencher 172 are optical tags included on the aptamer. The fluorescent tag 170 and the quencher 172 are configured to change an optical property of the aptamer. In some examples, the optical property is fluorescence. Alternatively, or in addition, a non-limiting list of other optical properties that may be changed by one ore more configured optical tags may be phosphorescence, optical absorbance magnitude, or peak wavelength for optical absorption. Typical optical aptamer probes may be used to detect complimentary DNA or RNA (as illustrated in FIG. 3C, or as not shown in FIG. 3C proteins, small molecules, toxins, or other solutes. The binding regions can be in portions of stems, loops, or other suitable regions of the aptamer and the examples shown herein are not limiting to the type of analyte and aptamer interactions that are possible for the present invention. Alternatively, or in addition to the analyte binding to the aptamer to increase fluorescence observed by the optical detector, aptamers can be designed such that fluorescence is quenched without binding of the analyte 190 (as shown in FIG. 3C) or such that fluorescence is quenched with binding of the analyte (not shown).

In practice, light emission from optical source 120 will excite the fluorescent tags 170 on the aptamers. The fluorescence from the optical source 120 is dependent on how much analyte 190 binds to the aptamers. As a result of additional analyte 190 binding to the aptamers, the availability of the quencher 172 to suppress fluorescence from the fluorescent tag 170 is changed. The device 100 relies on diffusion of analyte 190 to/from the sample fluid 14 to the aptamers located in the sensor fluid 18. As shown in FIG. 1, the diffusion of analyte 190 from the sample fluid 14 may occur through the membrane 136, which may be permeable to the analyte 190. In an embodiment of the present invention, the sample fluid 14 is interstitial fluid and the device 100 is configured to be either fully or partially implanted in-vivo (e.g., in the dermis). In some embodiments, the analyte 190 itself is the fluorescence quencher 172 (e.g., NADH, FAD, elastin, collagen, tryptophan, keratin) and is configured to absorb the energy, or wavelengths of light, from the fluorescent tag 170 and emitting light at a wavelength different than the fluorescent tag 170.

With reference to FIG. 2, where like numerals refer to like features, an alternate embodiment of a device 200 in accordance with the principles of the present invention is shown. In this embodiment, the membrane 136 is optional, and accordingly is not shown in the embodiment of the invention of FIG. 2. In contrast to a membrane retaining the position of the optical source 220 and the optical detector 222, the aptamers are immobilized adjacent to the optical source 220 and optical detector. As shown in FIG. 2, an element 232 may be used to maintain the aptamers within operational proximity to the optical source 220 and optical detector 222 by placing the aptamers within the element 232. As a non-limiting example, element 232 could be a hydrogel or other porous material such as an acrylamide hydrogel and aptamers may be bound to the hydrogel using acrydite attachment chemistry. Additionally, in the example shown in FIG. 2, the element 232 acts as an isolation element in device 200 in that species included in sample fluid 14 must first permeate the element 232 prior to potentially being exposed to the optical source 220 or the optical detector 222.

As with the embodiment shown in FIG. 1, the embodiment shown in FIG. 2 includes a substrate 210 adjacent to the optical source 220 and the optical detector 222, and the substrate 210 assists in holding the optical source 220 and optical detector 222 in place during operation of the device 200. Without membrane protection in a biofluid, aptamers can be degraded by solutes such as proteases, and therefore in an exemplary embodiment of FIG. 2, the aptamers may be XNA oligonucleotides. The XNA oligonucleotides may enable even sensing of large analytes 190, such as proteins, as those large analytes 190 diffuse to the aptamers from the sample fluid 14 through the element 232.

Turning now to FIG. 3A, multiple example aptamer configurations are shown that can enable embodiments of the present invention using a single aptamer or a plurality of aptamers that may bind together along portions of their structure that are complimentary. One example (shown at the top center of FIG. 3A), is a hairpin aptamer. The operation of a hairpin aptamer is then shown in FIGS. 3B and 3C. In a closed state (without having bound to analyte 190), as shown in FIG. 3B, the aptamer includes a loop structure 180, stem structure 182, fluorescent tag 170, and quencher 172. Fluorescent tag 170 and quencher 172 are in proximity to one another at termini of stem structure 182, such that fluorescence is largely quenched. The loop structure 180 of aptamer is then designed to have a sequence that is complementary to sequence of target analyte 190. Thus, as shown in FIG. 3C, when target analyte is brought into proximity with the aptamer, such that the aptamer binds to the analyte 190, the loop structure 180 will change to non-loop configuration due to binding (as shown in FIG. 3C), which separates the fluorescent tag 170 and quencher 172 such that significant fluorescence is enabled (due to quencher 172 no longer being able to quench fluorescence).

As a non-limiting example, a typical optical aptamer probe is 25 nucleotides long. A portion of the 25 nucleotides, for example 15 nucleotides, are complementary to or have a strong binding affinity for the analyte target and do not base pair with one another, while the five nucleotides at each terminus are complementary to each other rather than having strong affinity to the target analyte. Accordingly, the aptamer is configured to bond with a target analyte 190, specifically at the portion of the aptamer that has a strong binding affinity for the target analyte 190. In response to the aptamer being bound to the target analyte 190, a distance between the fluorescent tag 170 and the quencher 172 increases, resulting in an increase in fluorescence to be detected by the optical detector 122 (shown in FIG. 3C). A typical molecular beacon structure can be divided in 4 parts: 1) a loop structure 180, an 18-30 base pair region that is complementary to or has strong binding affinity for the target analyte; 2) a stem structure 182 formed by the attachment to both termini of the loop of two short (5 to 7 nucleotide residues) oligonucleotides that are complementary to each other; 3) a 5′ fluorophore at the 5′ end of the aptamer, which has a fluorescent dye is covalently attached; and 4) a 3′ quencher (non-fluorescent) dye that is covalently attached to the 3′ end of the aptamer. When the aptamer is in closed loop shape, the quencher 172 resides in proximity to the fluorescent tag 170, in an example: fluorophore, which results in quenching the fluorescent emission of the latter. When the aptamer is open (e.g., caused by analyte binding) the fluorescence from the fluorescent tag 170 is not quenched by the quencher 172. Accordingly, the aptamer is configured to detect a difference in fluorescence depending on whether the fluorescence is quenched or not. In response to more analyte binding to the aptamer, there is a detectable increase in fluorescence and a decrease in quenching of fluorescence. A closed loop structure is not required for the present invention, as the fluorescent tag 170 and quencher 172 can experience an average change in a distance between the fluorescent tag 170 and the quencher 172 with analyte binding, similar to that taught in electrochemical aptamer sensors where a redox tag experiences an average change in distance to an electrode surface.

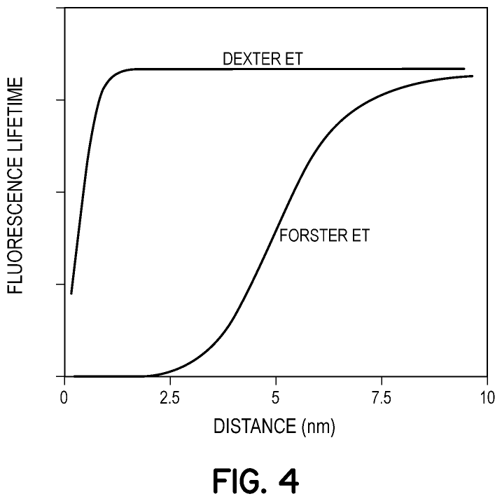

With reference to FIG. 4, fluorescence quenching is dominated by Forster energy transfer which has a distance dependence as also measurable by fluorescence lifetime. As shown in FIG. 4, fluorescence lifetime from the fluorescent tag 170 increases substantially as distance between the fluorescent tag 170 and the quencher 172 increases. This increase in fluorescent lifetime is detectable by the optical detector 122, and, as explained above, the distance between the fluorescent tag 170 and the quencher 172 is determined by whether the aptamer has bonded with the target analyte 190. An increase in analyte concentration in the sample fluid 14, will result in more binding to the aptamers in the sensor fluid 18, and accordingly a higher fluorescence detected by the optical detector 122, 222. In this way, the device 100, 200 is able to determine analyte 190 concentration or analyte 190 presence in the sample fluid 14.

Referring to FIGS. 1-4, embodiments of the present invention can be described in even greater detail. As membranes 136 are rarely perfect at excluding one species vs. another, membrane 136 may allow some aptamer to diffuse out of the device 100 into sample fluid 14. Furthermore, aptamers and/or fluorescent tags 170 can degrade over time. This loss of functional aptamer may be slow, but for longer lifetime devices 100, 200 (days, weeks, months), the loss of function may be significant. To cure the problem of degradation of the aptamers and/or fluorescent tags 170, the reservoir fluid 17 can continually diffuse in new aptamer to replace degraded or lost aptamers in the sensor fluid 18. As an example of an aptamer being degraded or lost, an aptamer 180 could be cleaved by a nuclease the fluorescent tag 170 resulting in the aptamer 180 giving a false ‘always on’ signal if the aptamer cleaving permanently separates the tag 170 and quencher 172. As a non-limiting example, sensor fluid 18 could have a volume of 1 μL, and reservoir fluid17 a volume of 500 μL, such that if sensor fluid 18, by itself, could enable sensing for a maximum of 3 days, the presence of the reservoir fluid 17, particularly, the reservoir fluid 17 including fresh aptamer to be supplied to the sensor fluid 18, sensing lifetime could be extended, instead, to 1500 days. To enable robust signal gain, the change in the aptamers' shape conformation upon analyte 190 binding may result in the fluorescent tag 170 and quencher 172 being changed in proximity to each other by at least 1 nm of distance and ideally brought within a distance of, in alternative embodiments, at least less than 10, 5, 2, or 1 nm for at least one of 10%, 20%, 40%, or 90% of the time. The significant change in distance from the fluorescent tag 170 and the quencher 172 as a result of the aptamer binding to the analyte is to ensure a strong signal change with analyte binding. Hairpin loop aptamers and other semi-stable aptamer configurations are preferred for this reason, and in many cases create a robust continuous sensor device 100, 200.

In addition, there are several other parameters that may be measured by the optical detector 122 other than fluorescence, yet the device 100, 200 may still operate as an optical sensor. For example, alternatively or in addition to fluorescence, to enable robust sensing in the presence of background fluorescence, fluorescence lifetime (fluorescence intensity vs. time), can instead be sensed, which as shown in FIG. 4, will change with fluorescent tag 170 and quencher 172 distance, resulting in a detection of analyte concentration. The lifetime of the fluorescent tag 170 can be at least 5, 10, 20, 50 and 100 ns to separate from background fluorescence which is typically <5 ns, which generally requires a optical detector 122, such as an optical detector, with a fast fall time (response time) that is less than 100 ns and ideally less than 10 ns. Fluorescence lifetime may reduce the need for calibration in long-lasting devices such as implantable devices. Fluorescence lifetime is independent of the quantity of aptamers, and/or degradation of the optical source 120, 220 or optical detector 122, 222. In another example, alternatively or in addition, the fluorescent tag 170 could be an excimer dye that results in a spectrum shift when brought adjacent to another excimer dye (e.g. same dyes at locations indicated as fluorescent tag 170 and quencher 172 in FIG. 3). Excimer and other dyes can also result in shifts in excitation cross-section, optical absorption peak, or magnitude based on distance between the dyes tagged on an aptamer. In addition, the fluorescence, optical absorption, or other properties of an optical dye, such as SYBR Green, can change if the dye is free in solution or intercalated in DNA, and the present invention can also use shape change of an aptamer to impact the optical properties of a single optical tag as the tag is brought closer to or further away from portions of an aptamer itself.

Furthermore, the environment surrounding the optical source 120, the optical detector 122, the fluorescent tag 170 or the quencher 172 could assist in the operation of detecting a parameter in the device 100, 200. For example, one or more surfaces of either the membrane 136 or the substrate 110, can be optically reflective to confine excitation and emission light from the optical source 120 or the fluorescent tag 170 to inside the device 100 and increase a signal-to-noise ratio of optical detection. Particularly, the membrane 136 could be a track-etch membrane coated with aluminum, or a 3M Vikuity ESR reflector with laser milled holes filled with a dense agar hydrogel, either approach providing >50% and ideally >90% reflectance.

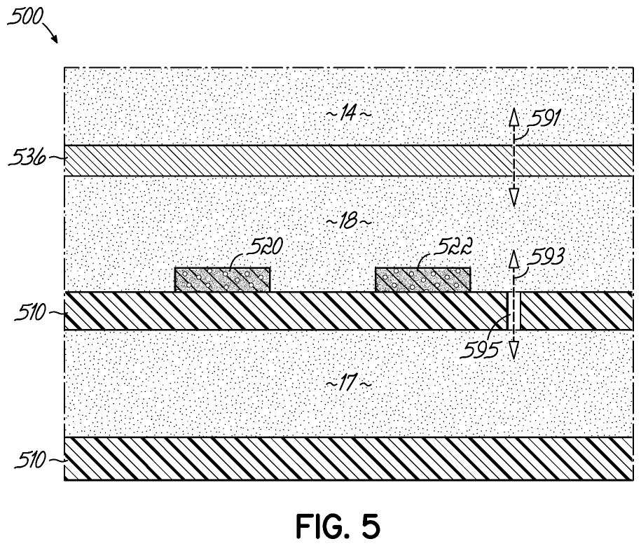

With reference to FIG. 5, where like numerals refer to like features, another embodiment in accordance with principles of the present invention is shown. In certain of the various embodiments discussed herein, a membrane is used to selectively allow passage of certain molecules and not of others. However, as no membrane is perfectly size selective, and as aptamers and redox tags can degrade over time, it may be advantageous to continually introduce a fresh supply of aptamers, solutes that increase performance of the sensor or improve longevity of the sensor (e.g. nuclease inhibitors, for example). Thus, as shown in FIG. 5, a portion of a device 500 includes substrates 510, an optical source 520, an optical detector 522, a membrane 536, a sample fluid 14, a sensor fluid 18, and a reservoir fluid 17. The membrane 536 exhibits mass flow represented at reference numeral 591, and the device also includes a diffusion restrictive feature 535 (such as a pinhole or membrane) with a mass flow represented at reference numeral 593.

As a nonlimiting example of that shown in FIG. 5, consider a 0.2 kDa dialysis membrane for membrane 536 and assume the aptamers are 10-100× larger than the solute to be detected (e.g., phenylalanine, cortisol, etc.). Assume the system is designed such that the volume of reservoir fluid 17 is at least one of 2×, 10×, 50×, or 250× greater than volume of sensor fluid 18 and that the mass flow 591 of aptamer is at least 2×, 10×, 50×, or 250× less than mass flow 593 of aptamer, while the mass flow 591 of the analyte is at least 2×, 10×, 50×, or 250× greater than the mass flow 593 of the analyte. As a result, the concentrations of analyte will be within at least 50%, 10%, 2%, or 0.4% of each other when comparing sample fluid 14 with sensor fluid 18, and the concentrations of aptamer will be within at least 50%, 10%, 2%, or 0.4% of each other when comparing sensor fluid 18 and reservoir fluid 17.

As a geometrical example, consider a membrane 536 with 0.2 cm² area and 10% porosity to the analyte, and a diffusion restrictive feature 535 that is a pinhole in materials 510 and 550 0.001 cm² in area and 0.001 cm in length. The mass transport for a small analyte through the membrane will be equivalent to 0.02 cm² area and the mass transport through the feature 535 0.001 cm², which is 20× different, satisfying the above stated criteria for design as shown in FIG. 5. As a result, both analyte and aptamer concentrations can be maintained for prolonged periods of times (days, weeks, months) even if aptamer is lost from the device or degraded over time. Aptamers could also degrade over time and their presence in the device and the presence of other contaminants such as nucleases or proteins could be problematic. For example, if signaling aptamers became cleaved and their molecular weight decreased, they could give a false higher reading of signal in embodiments of the present invention. With membrane protection of the sensor fluid from the sample fluid, most degradation or contamination modes will be very slow, such that the reservoir may also act as a waste removal element.

With further reference to FIG. 5, various aspects of the present invention are taught in greater detail with respect to isolation of aptamer (or the plurality of aptamers) in the sensor fluid 18 from the sample fluid 14 and reservoir fluid 17. The principles described with respect to FIG. 5 apply broadly to other embodiments of devices disclosed herein. In that regard, consider an aptamer sensor for creatinine or phenylalanine, (which have molecular weight of only 131 Da and 165 Da, respectively), and an aptamer having a molecular weight of 10-15 kDa (which is common for many aptamers). In such a situation, a membrane 536 having a 150 Da molecular weight cutoff could be used to prevent movement of aptamers from sensor fluid to another fluid (like sensor fluid to sample fluid). Alternatively, commercial membranes such as Dow FilmTech Polyamide membranes with 200-400 Da cutoffs may be suitable for use as membrane 536. Further still, non-limiting examples of alternate materials for the membrane 536 include cellulose acetate, polypiperazine-amide, and polydimethylsiloxane. But even in such a case, conventional membranes are designed for pressure-driven separations, and so include a significant thickness or a backing layer, which increases overall thickness (on the order of 100s of μM). A thick membrane (100's to 1000's of μm), such as these would impart a penalty on device response time to changes in analyte concentration, as the analyte must diffuse through the thick membrane (a thicker membrane 36 not only creates a more tortuous path for analyte diffusion, but it increases the distances over which the analyte concentration gradient exists between sample and sensor which further decreases the diffusive flux). Therefore, in one embodiment of the present invention the membrane backing material can be facing the sample fluid 14 to resolve this lag time increase at least in part because backing material typically has high porosity.

As an additional example, consider phenylalanine and a ˜90 nm thick epoxy membrane 536 with an effective diffusion coefficient divided by membrane 536 thickness of Deff/Δx of between about 5 m s⁻¹×10⁻³ and 0.005 m s⁻¹×10⁻³, as taught by Rodler et al. in Freestanding ultrathin films for separation of small molecules in an aqueous environment, Journal of Biotechnology, Volume 288, Dec. 20, 2018, pages 48-54. (https://doi.org/10.1016/j.jbiotec.2018.10.002). The thickness or porosity of the membrane 536 can be adjusted easily. Accordingly, the present invention may benefit from a membrane 536 that has a Deff/Δx of, in one embodiment, at least 5 m s⁻¹×10⁻³. In another embodiment, the Deff/Δx is at least 0.5 m s⁻¹×10⁻³. In yet another embodiment, the Deff/Δx is at least 0.05 m s⁻¹×10⁻³. In another embodiment, the Deff/Δx is at least 0.005 m s⁻¹×10⁻³. Membranes 536 of the present invention are, in one embodiment, less than about 100 nm thick. In another embodiment, membranes of the present invention are less than 1 μm thick. In yet another embodiment, membranes of the present invention are less than 10 μm thick. In another embodiment, membranes of the present invention are less than 100 μm thick. A membrane 536 with a well-designed Deff/Δx of at least 5 m s⁻¹×10⁻³ and thickness of sensor fluid 18 of ˜1-10 μm, can enable a device on/off time for the sensor to measure 90% of the sample fluid concentration in at least <15 min.

Additionally, in some cases, the analyte molecular weight will become larger or be too large to permeate the membrane 536, and the aptamer might permeate the membrane 536 due to its molecular weight or due to a stranded geometry that allows it to navigate through a membrane similar to a rope being pulled or pushed through a screen (if the rope were balled up, it could not be pushed through the screen). Therefore, alternate methods of isolating the aptamer from sample fluid 14 are needed in some cases. One important factor is the retention % vs. molecular weight, (see the graph for a membrane 536 as illustrated in FIG. 4). This is also referred to as retentivity. A typical membrane 536 can provide >90% retention for example at 10 kDa, and <9% retention at <1 kDa for a change in retentivity/change in molecular weight of ˜1×. This is not highly selective with respect to the present invention, because for example, with an implanted device, aptamers could be slowly and continually lost over time. Therefore, the present invention may benefit from a membrane with a change in retentivity/change in molecular weight that, in one embodiment, is at least 2×. In another embodiment, the membrane has a change in retentivity/change in molecular weight that is at least 5×. In yet another embodiment, the membrane has a change in retentivity/change in molecular weight that is at least 10×. In one embodiment, the membrane has a change in retentivity/change in molecular weight that is at least 20×.

Further, as taught in other embodiments, while some aptamer may be continually lost from the sensor fluid, fresh aptamer can diffuse in from an adjacent reservoir to replenish lost aptamer. This reservoir is shown in FIG. 5 as including reservoir fluid 17. As an example, the volume of sensor fluid 18 could be 1 μL in volume using an area of membrane 536 of 0.1 cm² and a separation distance between membrane 536 and optical source 520 and optical detector 522 of 0.01 cm (100 μm). A reservoir, including reservoir fluid 17, which in turn includes fresh aptamer solution could be in fluid communication with the volume including sensor fluid 18 via a pore 595 that is only 0.001 cm², such that analyte and aptamer would very slowly diffuse into/out of the aptamer reservoir, again, shown in FIG. 5 as including reservoir fluid 17, but such that new aptamer would constantly diffuse into the volume including the sensor fluid 18 as any aptamer is lost from the sensor fluid 18 through the membrane 536 to the sample fluid 14. In one embodiment, the pore 595 is a fluidic connection from the reservoir, including the reservoir fluid 17, and the volume, including the sensor fluid 18.

Such an approach could allow for protein sensing. For example, assume a sensor fluid 18 volume of 100 nL, and a membrane 536 that retains 90% of the aptamer over 6 hours, and which can allow a protein such as luteinizing hormone to diffuse into the sensor fluid 18 and achieve 90% of sample fluid 14 concentration of the hormone within 12 hrs. This would allow a device 500 to sufficiently measure luteinizing hormone for fertility monitoring applications. Now, if the reservoir including reservoir fluid 17 with aptamer had a volume of 200 μL, then it could lose 10% of its aptamer before a sensor signal would be impacted by 10%. If the device 500 is losing aptamer through the membrane 536 at a rate of, for example, 10% of aptamer every 6 hours in the 100 nL volume including sensor fluid 18, then with the 200 μL reservoir including reservoir fluid 17, the device 500 could last 2000× longer or 12,000 hours or >16 months, more than long enough for creating an implantable device 500. Therefore, depending on the volume of the reservoir including reservoir fluid 17 and scaling of other device 500 dimensions and membrane 536 porosity, the present invention can retain 90% of the initial aptamer concentration in the sensor fluid 18 for at least >16 months, >8 months, >4 months, >2 months, >1 month, >2 weeks or >1 week.

Further, and as will now be described in greater detail, there is no major penalty if the aptamer is designed such that one end of the aptamer is inactive and increases the total molecular weight of the aptamer by at least 50%. For example, in some embodiments, the aptamer includes an active end configured to bind to the analyte and which has the redox tag. In some embodiments, the aptamer may include a longer inactive end configured to provide molecular weight or size to the aptamer and/or configured to reduce aptamer permeation through the membrane 536. The longer inactive end may be configured to be rigid or have at least one permanent fold, wherein the rigid aptamer or aptamer including a permanent fold is dimensionally larger than a non-rigid aptamer or aptamer not including a permanent fold. In one embodiment, the molecular weight of such aptamers is at least >15 kDa. In another embodiment, the molecular weight of such aptamers is at least >30 kDa. In yet another embodiment, the molecular weight of such aptamers is at least >60 kDa. In one embodiment, the molecular weight of such aptamers is at least >120 kDa. For example, that active end aptamer could have a molecular weight of at least <20, <10, or <5 kDa, and the inactive end of the aptamer may be configured to be folded and therefore configured to increase the total size and molecular weight of the aptamer.

In additional embodiments, aptamers may be attached to other materials, or to nanoparticles, to also help isolate them from the sample fluid. For example, an aptamer could be attached to a polyethylene glycol polymer, the polyethylene glycol polymer may have a molecular weight of about 300 kDa, which can be referred to as a ‘particle’. Particles could be other polymers, metal such as gold, carbon, or iron-oxide and can be, in different embodiments, at least >1 nm, >3 nm, >10 nm, >30 nm, or >100 nm in diameter and still stably dispersed in solution as is known using one or more methods like those used in the art of pigment and nanodispersions. Aptamers can be bound to iron nanoparticles using, as a non-limiting example, dibromomaleimide (DBM)-tennination, and bound to gold nanoparticles using thiol termination. With use of magnetic nanoparticles such as iron-oxide, the aptamer isolation element may also be a magnet that retains the nanoparticles near the membrane with or without use of a membrane, and in this example the aptamer isolation element is a magnet. This approach could allow the present invention to measure a protein analyte, for example a 30 kDa protein with an average diameter of <5 nm.

Consider an example that teaches the impact of device parameters on device lag time. Assume a device that operates with a sample fluid that is interstitial fluid and where ˜1 μM of fluorescently tagged aptamer is used to provide a safe margin on tag signal strength vs. background fluorescence interferents. If the distance between the membrane and electrode was 5 μm, then for cortisol at 10 nM and 1 μM aptamer the ‘equivalent’ volume of sensor fluid is 100× greater or 500 μm thick from a lag time perspective. Next, assume a membrane that is 10% porous to cortisol, for this configuration, the cortisol can diffuse into the sensor fluid and reach 90% of its concentration in the sample fluid in less than 20 minutes. Next, utilize a membrane that is 1.66% or 50% porous to cortisol, and the lag time becomes 60 min or as little as 4 min, respectively. Aptamer concentration can be increased or decreased to adjust this lag time, the distance between the membrane and electrode and/or substrate can be modified to adjust this lag time by adjusting the sensor fluid volume, and for different analytes a higher or lower analyte concentration will also adjust this lag time (e.g., cortisol at 1 nM will have 10× greater lag time while cortisol at 100 nM would have 10× lesser lag time. Therefore generally, the present invention can enable devices with lag times to reach 90% of sensor response that are less than at least one of 180 min, 60 min, 20 min, 5 min, 2 min. The challenges with reduced sample volumes for optical detection include shorter optical detection path lengths, and therefore detection volumes could instead be primarily the space in between elements 120, 122, as illustrated in FIG. 1 (e.g., the spacing could be 10's, 100's, or 1000's of μm or even longer), which would still allow a small sensor fluid volume or thickness next to the membrane. The specific geometries illustrated and taught herein are non-limiting, and simply are used to teach generally the principles of embodiments of the invention.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A device for detecting the presence of, or measuring the concentration or amount of, at least one analyte in a sample fluid, the device comprising:
a sensor fluid;
a plurality of aptamers disposed in the sensor fluid, one or more aptamers of the plurality of aptamers configured to bind to an analyte, each of the one or more aptamers comprising:
at least one optical tag, wherein the optical tag is configured to provide a change in at least one optical property of the aptamer between a first state in which the aptamer is bound to the analyte and a second state in which the aptamer is not bound to the analyte, the first state and the second state differing in the shape of the aptamer;
at least one isolation element retaining the aptamer in the sensor fluid;
an optical source configured to emit light, the optical source in communication with the sensor fluid; and
an optical detector configured to detect a change in at least one optical property of the aptamers.

2. The device of claim 1 wherein, a first optical tag is a fluorescent tag configured to emit an amount of light, and a second optical tag is a quencher configured to quench at least a portion of the light emitted by the first optical tag.

3. The device of claim 1, wherein the isolation element is a membrane.

4. The device of claim 1, wherein the isolation element is a hydrogel, wherein the one or more aptamers are bound to the hydrogel.

5. The device of claim 2, wherein the quencher is at least less than one of 10 nm, 5 nm, 2 nm, or 1 nm from the fluorescent tag for at least one of 10%, 20%, 40%, or 90% of the time during the operation of the device.

6. The device of claim 2, wherein the optical detector is configured to detect a fluorescence lifetime from the fluorescence of the light emitted by the fluorescent tag.

7. The device of claim 6, wherein the fluorescence lifetime is at least one of less than 5 ns, 10 ns, 20 ns, 50 ns, and 100 ns

8. The device of claim 6, wherein the optical detector has a response time that is less than 10 ns or less than 100 ns.

9. The device of claim 1, wherein one or more surfaces of the optical device, the optical detector, or the substrate that are in communication with the sensor fluid are >50% reflective.

10. The device of claim 1, further comprising a reservoir fluid that is in fluidic communication with the sensor fluid.

11. The device of claim 10, wherein a volume of the reservoir fluid is at least one of 2×, 10×, 50×, or 250× greater than a volume of the sensor fluid.

12. The device of claim 10, wherein a first mass flow of aptamer through the isolation element and a second mass flow of aptamer through the fluidic connection between the sensor fluid and the reservoir fluid, and the first mass flow is at least 2×, 10×, 50×, or 250× less than the second mass flow.

13. The device of claim 10, wherein there is a first mass flow of analyte through the isolation element and a second mass flow of analyte through the fluidic connection between the sensor fluid and the reservoir fluid, and the first mass flow is at least 2×, 10×, 50×, or 250× greater than the second mass flow.

14. The device of claim 1, wherein a concentration of the plurality of aptamers in the sensor fluid is within at least 50%, 10%, 2%, or 0.4% of a concentration of the plurality of aptamers in the reservoir fluid.

15. The device of claim 1, wherein a concentration of analyte in the sensor fluid is within at least 50%, 10%, 2%, or 0.4% of a concentration of analyte in the sample fluid.

16. The device of claim 3, wherein the membrane has a backing material, and the backing material is in communication with the sample fluid.

17. The device of claim 10, wherein the reservoir fluid is adapted to absorb from the sensor fluid one or more aptamers of the plurality of aptamers that have degraded or absorb from the sensor fluid any optical tag that has degraded.

18. The device of claim 3, wherein the membrane has a Deff/Δx that is greater than an amount selected from the group consisting of 5 m s⁻¹×10⁻³, 0.5, 0.05 m s⁻¹×10⁻³, and 0.005 m s⁻¹×10⁻³.

19. The device of claim 3, wherein the membrane has a thickness that is less than an amount selected from the group consisting of 100 nm, 1 μm, 10 μm, and 100 μm.

20. The device of claim 3, wherein the membrane has retentivity/molecular weight that is greater than an amount selected from the group consisting of 2×, 5×, 10×, and 20×.

21. The device of claim 10, wherein the initial amount of aptamers in the plurality of aptamers disposed in the sensor fluid provides an initial aptamer concentration in the sensor fluid, and wherein the device is configured to retain 90% of the initial aptamer concentration in the sensor fluid for a period of time selected from the group consisting of >16 months, >8 months, >4 months, >2 months, >1 month, >2 weeks, and >1 week.

22. The device of claim 1, wherein one or more aptamers of the plurality of aptamers each includes an active portion and an inactive portion, wherein the inactive portion increases the total aptamer molecular weight by at least 50%.

23. The device of claim 22, wherein the inactive portion is rigid.

24. The device of claim 22, wherein the inactive portion includes at least one permanent fold.

25. The device of claim 3, wherein the molecular weight of each aptamer of the plurality of aptamers is an amount selected from the group consisting of >15 kDa, >30 kDa, >60 kDa and >120 kDa.

26. The device of claim 22, wherein the active portion has a molecular weight that is selected from the group consisting of <20 kDa, <10 kDa, and <5 kDa.

27. The device of claim 3, wherein a majority of the plurality of aptamers are bound to a plurality of particles.

28. The device of claim 27, wherein the size of the particles is selected from the group consisting of >1 nm, >3 nM, >10 nM, >30 nM, and >100 nM in diameter.

29. The device of claim 1, wherein the analyte is a small molecule.

30. The device of claim 1, wherein the analyte is a protein.

31. The device of claim 1, wherein the device has a lag time and the lag times to reach 90% of sensor response is less than at least one of 180 min, 60 min, 20 min, 5 min, and 2 min.

32. The device of claim 1, wherein the device further comprising a surface opposite of the isolation element that defines the sensor fluid volume and the distance between said surface and said element is at least one of less than 100 μm, 10 μm, 1 μm, 0.1 μm, and 0.01 μm.

33. The device of claim 1, wherein one or more aptamers of the plurality of aptamers are folded aptamers.

34. The device of claim 1, wherein one or more aptamers of the plurality of aptamers are unfolded aptamers.

35. The device of claim 2, wherein one or more aptamers of the plurality of aptamers are configured to bind to the analyte such that the analyte separates the fluorescent tag and a quencher.

36. The device of claim 2, wherein the fluorescent tag is an excimer dye.

37. The device of claim 3, wherein the membrane is a dialysis membrane.

38. The device of claim 1, wherein the aptamer is 10×-100× larger than the analyte.

39. The device of claim 3, wherein the membrane comprises a feature, the feature sized to permit a mass transport therethrough 20× smaller than a surface area of the membrane.

40. The device of claim 3, wherein the device is configured to measure 90% of the sample fluid concentration in at least <15 min.

41. The device of claim 27, wherein the particles are selected from a group consisting of a polymer, a metal, a carbon, and an iron-oxide.

42. The device of claim 41, wherein each particle is at least >1 nm, >3 nm, >10 nm, >30 nm, or >100 nm in diameter.

43. The device of claim 41, wherein the particle is a magnetic nanoparticle and the isolation element is a magnet.

44. A method of detecting the presence of, or measuring the amount or concentration of, an analyte in a sample fluid, the method comprising:
bringing a sample fluid into contact with a plurality of aptamers in a sensor fluid, one or more aptamers of the plurality of aptamers comprising:
at least one optical tag, wherein the optical tag is configured to provide a change in at least one optical property of the aptamer between a first state in which the aptamer is bound to the analyte and a second state in which the aptamer is not bound to the analyte, the first state and the second state differing in the shape of the aptamer;
at least one isolation element retaining the aptamer in the sensor fluid;
an optical source configured to emit light, the optical source in communication with the sensor fluid; and
an optical detector configured to detect a change in at least one optical property of the aptamers; and
detecting any change in optical property of one or more aptamers of the plurality of aptamers.

45. The method of claim 44, wherein a first optical tag is a fluorescent tag configured to emit an amount of light, and a second optical tag is a quencher configured to quench at least a portion of the light emitted by the first tag.

46. The method of claim 44, wherein the sensor fluid is in fluid communication with a reservoir fluid, the method further comprising flowing a first mass flow of aptamer through an isolation element and a second mass flow of aptamer through a fluidic connection between the sensor fluid and the reservoir fluid, and the first mass flow is at least 2×, 10×, 50×, or 250× less than the second mass flow.

47. The method of claim 44, wherein the sensor fluid is in fluid communication with a reservoir fluid, the method further comprising flowing a first mass flow of aptamer through an isolation element and a second mass flow of aptamer through a fluidic connection between the sensor fluid and the reservoir fluid, and the first mass flow is at least 2×, 10×, 50×, or 250× greater than the second mass flow.

48. The method of claim 44, wherein the sensor fluid is in fluid communication with a reservoir fluid, the method further comprising transporting the aptamer that has degraded or fluorescent tag that has degraded from the sensor fluid to the reservoir fluid.

49. The method of claim 44, wherein the sensor fluid is in fluid communication with a reservoir fluid, the method further comprising transporting a second plurality of aptamers from the reservoir fluid to the sensor fluid.
